(19) United States
(12) Patent Application Publication
(10) Pub. No.: US 2003/0166884 A1
(43) Pub. Date: Sep. 4, 2003

(54) Nucleotide sequences which code for the rpoB gene

(75) Inventors: Bettina Moeckel, Duesseldorf (DE); Brigitte Bathe, Salzkotten (DE); Thomas Hermann, Bielefeld (DE); Walter Pfefferle, Halle (DE); Michael Binder, Steinhagen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/076,406

(22) Filed: Feb. 19, 2002

Related U.S. Application Data
(63) Continuation-in-part of application No. 09/887,052, filed on Jun. 25, 2001.

(30) Foreign Application Priority Data
Feb. 16, 2001 (DE) 101 07 229.5

(51) Int. Cl. C07H 21/02; C07H 21/04

(52) U.S. Cl. 536/23.1

(57) ABSTRACT

The present invention relates to polynucleotides corresponding to the rpoB gene and which encode the β-subunit of RNA polymerase B, methods of producing L-amino acids, and methods of screening for polynucleotides which encode proteins having activity of the β-subunit of RNA polymerase B.

### CROSS-REFERENCE TO RELATED APPLICATION

The present application claims priority to German Application No. DE10107229.5 filed Feb. 16, 2001, the entire contents of which are incorporated herein by reference.

### BACKGROUND OF THE INVENTION

1. Field of the Invention

The present invention relates to polynucleotides corresponding to the rpoB gene and which encode the β-subunit of RNA polymerase B, methods of producing L-amino acids, and methods of screening for polynucleotides which encode proteins having activity of the β-subunit of RNA polymerase B.

2. Discussion of the Background

L-amino acids, especially L-lysine, are used in human medicine and in the pharmaceuticals industry, in the foodstuffs industry and, very especially, in the feeding of animals.

It is known that amino acids are produced by fermentation of strains of coryneform bacteria, especially Corynebacterium glutamicum. Because of their great importance, attempts are continuously being made to improve the production processes. Improvements to the processes may concern measures relating to the fermentation, such as, for example, stirring and oxygen supply, or the composition of the nutrient media, such as, for example, the sugar concentration during the fermentation, or working up to the product form by, for example, ion-exchange chromatography, or the intrinsic performance properties of the microorganism itself.

In order to improve the performance properties of such microorganisms, methods of mutagenesis, selection and mutant selection are employed. Such methods yield strains which are resistant to antimetabolites or are auxotrophic for metabolites that are important in terms of regulation, and which produce amino acids.

For a number of years, methods of recombinant DNA technology have also been used for improving the strain of L-amino acid-producing strains of Corynebacterium, by amplifying individual amino acid biosynthesis genes and studying the effect on amino acid production.

However, there remains a critical need for improved methods of producing L-amino acids and thus for the provision of strains of bacteria producing higher amounts of L-amino acids. On a commercial or industrial scale even small improvements in the yield of L-amino acids, or the efficiency of their production, are economically significant. Prior to the present invention, it was not recognized that enhancement of the rpoB gene encoding the β-subunit of RNA polymerase B would improve L-amino acid yields.

### SUMMARY OF THE INVENTION

One object of the present invention, is providing a new process adjuvant for improving the fermentative production of L-amino acids, particularly L-lysine and L-glutamate. Such process adjuvants include enhanced bacteria, preferably enhanced Coryneform bacteria which express enhanced levels of the β-subunit of RNA polymerase B which is encoded by the rpoB gene.

Thus, another object of the present invention is providing such a bacterium, which expresses enhanced amounts of the β-subunit of RNA polymerase B or gene products of the rpoB gene.

Another object of the present invention is providing a bacterium, preferably a Coryneform bacterium, which expresses a polypeptide that has an enhanced β-subunit of RNA polymerase B activity.

Another object of the invention is to provide a nucleotide sequence encoding a polypeptide which has a β-subunit of RNA polymerase B sequence. One embodiment of such a sequence is the nucleotide sequence of SEQ ID NO: 1. Other embodiments of such a sequence is the nucleotide sequences of SEQ ID NOS:3 and 5.

A further object of the invention is a method of making a β-subunit of RNA polymerase B or an isolated polypeptide having a β-subunit of RNA polymerase B activity, as well as use of such isolated polypeptides in the production of amino acids. One embodiment of such a polypeptide is the polypeptide having the amino acid sequence of SEQ ID NO: 2. Other embodiments of such a sequence is the amino acid sequence of SEQ ID NOS:4 and 6.

In one embodiment the invention provides isolated polypeptides comprising the amino acid sequences in SEQ ID NOS:2, 4 and/or 6.

Other objects of the invention include methods of detecting nucleic acid sequences homologous to SEQ ID NO: 1, particularly nucleic acid sequences encoding polypeptides that have the activity of a β-subunit of RNA polymerase B, and methods of making nucleic acids encoding such polypeptides.

The above objects highlight certain aspects of the invention. Additional objects, aspects and embodiments of the invention are found in the following detailed description of the invention.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

Where L-amino acids or amino acids are mentioned hereinbelow, they are to be understood as meaning one or more amino acids, including their salts, selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine is especially preferred.

Where L-lysine or lysine is mentioned hereinbelow, it is to be understood as meaning not only the bases but also the salts, such as, for example, lysine monohydrochloride or lysine sulfate.

The invention provides an isolated polynucleotide from coryneform bacteria, containing a polynucleotide sequence coding for the rpoB gene, selected from the group

a) polynucleotide that is at least 70% identical with a polynucleotide that codes for a polypeptide containing the amino acid sequence of SEQ ID No. 2,

b) polynucleotide that codes for a polypeptide containing an amino acid sequence that is at least 70% identical with the amino acid sequence of SEQ ID No. 2,

c) polynucleotide that is complementary to the polynucleotides of a) or b), and

d) polynucleotide containing at least 15 consecutive nucleotides of the polynucleotide sequence of a), b) or c),

the polypeptide preferably exhibiting the activity of the β-subunit of RNA polymerase B.

The invention also provides the above-mentioned polynucleotide, it preferably being a replicatable DNA containing:

(i) the nucleotide sequence shown in SEQ ID No. 1, or

(ii) at least one sequence that corresponds to sequence (i) within the region of the degeneracy of the genetic code, or

(iii) at least one sequence that hybridizes with the sequence that is complementary to sequence (i) or (ii), and optionally

(iv) sense mutations in (i) which are neutral in terms of function and which do not change the activity of the protein/polypeptide.

Finally, the invention also provides polynucleotides selected from the group

a) polynucleotides containing at least 15 consecutive nucleotides selected from the nucleotide sequence of SEQ ID No. 1 between positions 1 and 701

b) polynucleotides containing at least 15 consecutive nucleotides selected from the nucleotide sequence of SEQ ID No. 1 between positions 702 and 4199

c) polynucleotides containing at least 15 consecutive nucleotides selected from the nucleotide sequence of SEQ ID No. 1 between positions 4200 and 5099.

The invention also provides

a replicatable polynucleotide, especially DNA, containing the nucleotide sequence as shown in SEQ ID No. 1;

a polynucleotide that codes for a polypeptide containing the amino acid sequence as shown in SEQ ID No. 2;

a vector containing the polynucleotide of the invention, especially a shuttle vector or a plasmid vector, and

coryneform bacteria which contain the vector or in which the rpoB gene has been enhanced.

The invention also provides polynucleotides consisting substantially of a polynucleotide sequence, which are obtainable by screening, by means of hybridization, a corresponding gene library of a coryneform bacteria that contains the complete gene or parts thereof, using a probe containing the sequence of the polynucleotide of the invention according to SEQ ID No. 1 or a fragment thereof, and isolating the mentioned polynucleotide sequence.

Polynucleotides that contain the sequences of the invention are suitable as hybridization probes for RNA, cDNA and DNA, in order to isolate in their complete length nucleic acids or polynucleotides or genes that code for the β-subunit of RNA polymerase B, or in order to isolate nucleic acids or polynucleotides or genes that are very similar to the sequence of the rpoB gene. They are likewise suitable for incorporation into so-called “arrays”, “micro arrays” or “DNA chips” in order to detect and determine the corresponding polynucleotides.

Polynucleotides that contain the sequences of the invention are also suitable as primers, with the aid of which it is possible, by means of the polymerase chain reaction (PCR), to produce DNA of genes that code for the β-subunit of RNA polymerase B.

Such oligonucleotides acting as probes or primers contain at least 25, 26, 27, 28, 29 or 30, preferably at least 20, 21, 22, 23 or 24, very especially preferably at least 15, 16, 17, 18 or 19, consecutive nucleotides. Also suitable are oligonucleotides having a length of at least 31, 32, 33, 34, 35, 36, 37, 38, 39 or 40 or of at least 41, 42, 43, 44, 45, 46, 47, 48, 49 or 50 nucleotides. Oligonucleotides having a length of at least 100, 150, 200, 250 or 300 nucleotides may also be suitable.

“Isolated” means removed from its natural environment.

“Polynucleotide” generally refers to polyribonucleotides and polydeoxyribonucleotides, it being possible for the RNA or DNA to be unmodified or modified.

The polynucleotides of the invention include a polynucleotide according to SEQ ID No. 1 or a fragment prepared therefrom, and also polynucleotides that are at least especially from 70% to 80%, preferably at least from 81% to 85%, especially preferably at least from 86% to 90%, and very especially preferably at least 91%, 93%, 95%, 97% or 99%, identical with the polynucleotide according to SEQ ID No. 1, or with a fragment prepared therefrom.

“Polypeptides” are to be understood as being peptides or proteins that contain two or more amino acids bonded via peptide bonds.

The polypeptides of the invention include a polypeptide according to SEQ ID No. 2, especially those having the biological activity of the β-subunit of RNA polymerase B, and also those that are at least from 70% to 80%, preferably at least from 81% to 85%, especially preferably at least from 86% to 90%, and very especially preferably at least 91%, 93%, 95%, 97% or 99%, identical with the polypeptide according to SEQ ID No. 2 and exhibit the mentioned activity.

The invention also provides a process for the production, by fermentation, of amino acids selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, using coryneform bacteria which, in particular, already produce amino acids and in which the nucleotide sequences coding for the rpoB gene are enhanced, especially overexpressed.

The term “enhancement” in this connection describes the increasing of the intracellular activity of one or more enzymes or proteins in a microorganism that are coded for by the corresponding DNA, by, for example, increasing the number of copies of the gene or genes, using a strong promoter or using a gene or allele that codes for a corresponding enzyme or protein having a high level of activity, and optionally by combining those measures.

The microorganisms provided by the present invention can produce L-amino acids from glucose, saccharose, lactose, fructose, maltose, molasses, starch, cellulose or from glycerol and ethanol. They may be representatives of coryneform bacteria, especially of the genus Corynebacterium. In the case of the genus Corynebacterium, special mention may be made of the species Corynebacterium glutamicum, which is known to those skilled in the art for its ability to produce L-amino acids.

Suitable strains of the genus Corynebacterium, especially of the species Corynebacterium glutamicum (C. glutamicum), are especially the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

and L-amino acid-producing mutants or strains prepared therefrom, such as, for example, the L-lysine-producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464

Corynebacterium glutamicum DM58-1

Corynebacterium glutamicum DG52-5

Corynebacterium glutamicum DSM5714 and

Corynebacterium glutamicum DSM12866.

Preferably, a bacterial strain with attenuated expression of a rpoB gene that encodes a polypeptide with activity of the βunit of RNA polymerase B will improve amino acid yield at least 1%.

The inventors have succeeded in isolating the new rpoB gene of C. glutamicum which codes for the β-subunit of RNA polymerase B, which is a β-subunit of RNA polymerase B.

In order to isolate the rpoB gene or other genes from C. glutamicum, a gene library of that microorganism in Escherichia coli (E. coli) is first prepared. The preparation of gene libraries is written down in generally known textbooks and handbooks. There may be mentioned as an example the textbook of Winnacker: Gene und Klone, Eine Einführung in die Gentechnologie (Verlag Chemie, Weinheim, Germany, 1990) or the handbook of Sambrook et al.: Molecular Cloning, A Laboratory Manual (Cold Spring Harbor Laboratory Press, 1989). A very well known gene library is that of the E. coli K-12 strain W3110, which has been prepared by Kohara et al. (Cell 50, 495-508 (1987)) in λ-vectors. Bathe et al. (Molecular and General Genetics, 252:255-265, 1996) describe a gene library of C. glutamicum ATCC13032, which has been prepared with the aid of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:1563-1575).

Börmann et al. (Molecular Microbiology 6(3), 317-326) (sic) (1992)) in turn describe a gene library of C. glutamicum ATCC13032 using the cosmid pHC79 (Hohn and Collins, Gene 11, 291-298 (1980)).

For the preparation of a gene library of C. glutamicum in E. coli it is also possible to use plasmids such as pBR322 (Bolivar, Life Sciences, 25, 807-818 (1979)) or pUC9 (Vieira et al., 1982, Gene, 19:259-268). Suitable hosts are especially those E. coli strains that are restriction- and recombination-defective. An example thereof is the strain DH5αmcr, which has been described by Grant et al. (Proceedings of the National Academy of Sciences USA, 87 (1990) 4645-4649). The long DNA fragments cloned with the aid of cosmids can then in turn be subcloned into customary vectors suitable for sequencing and then sequenced, as is described, for example, in Sanger et al. (Proceedings of the National Academy of Sciences of the United States of America, 74:5463-5467, 1977).

The resulting DNA sequences can then be studied using known algorithms or sequence-analysis programs, such as, for example, that of Staden (Nucleic Acids Research 14, 217-232 (1986)), that of Marck (Nucleic Acids Research 16, 1829-1836 (1988)) or the GCG program of Butler (Methods of Biochemical Analysis 39, 74-97 (1998)).

The novel DNA sequence of C. glutamicum coding for the rpoB gene has been found and, as SEQ ID No. 1, forms part of the present invention. Furthermore, the amino acid sequence of the corresponding protein has been derived from the present DNA sequence using the methods described above. The resulting amino acid sequence of the rpoB gene product is shown in SEQ ID No. 2. It is known that enzymes belonging to the host are able to cleave the N-terminal amino acid methionine or formylmethionine of the protein that is formed.

Coding DNA sequences that result from SEQ ID No. 1 by the degeneracy of the genetic code also form part of the invention. Likewise, DNA sequences that hybridize with SEQ ID No. 1 or parts of SEQ ID No. 1 form part of the invention. Furthermore, to those skilled in the art, conservative amino acid substitutions, such as, for example, the substitution of glycine with alanine or of aspartic acid with glutamic acid, in proteins are known as sense mutations, which do not lead to any fundamental change in the activity of the protein, that is to say are neutral in terms of function. Such mutations are known inter alia also as neutral substitutions. It is also known that changes at the N- and/or C-terminus of a protein do not substantially impair its function or may even stabilise it. The person skilled in the art will find relevant information inter alia in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences that result in a corresponding manner from SEQ ID No. 2 likewise form part of the invention.

Similarly, DNA sequences that hybridize with SEQ ID No. 1 or parts of SEQ ID No. 1 form part of the invention. Finally, DNA sequences that are produced by the polymerase chain reaction (PCR) using primers that result from SEQ ID No. 1 form part of the invention. Such oligonucleotides typically have a length of at least 15 nucleotides.

The person skilled in the art will find instructions on the identification of DNA sequences by means of hybridization inter alia in the handbook “The DIG System Users Guide for Filter Hybridization” from Boehringer Mannheim GmbH (Mannheim, Germany, 1993) and in Liebl et al. (International Journal of Systematic Bacteriology (1991) 41: 255-260). The hybridization takes place under stringent conditions, that is to say there are formed only hybrids in which the probe and the target sequence, i.e. the polynucleotides treated with the probe, are at least 70% identical. It is known that the stringency of the hybridization, including the washing steps, is influenced or determined by varying the buffer composition, the temperature and the salt concentration. The hybridization reaction is preferably carried out with relatively low stringency as compared with the washing steps (Hybaid Hybridisation Guide, Hybaid Limited, Teddington, UK, 1996).

There may be used for the hybridization reaction, for example, a 5×SSC buffer at a temperature of approximately from 50° C. to 68° C. In that case, probes may also hybridize with polynucleotides that are less than 70% identical with the sequence of the probe. Such hybrids are less stable and are removed by washing under stringent conditions. That may be achieved, for example, by lowering the salt concentration to 2×SSC and optionally subsequently to 0.5×SSC (The DIG System User's Guide for Filter Hybridisation, Boehringer Mannheim, Mannheim, Germany, 1995), a temperature of approximately from 500C to 68° C. being set. It is optionally possible to lower the salt concentration down to 0.1×SSC. By raising the hybridization temperature stepwise from 50° C. to 68° C. in steps of approximately from 1 to 2° C., it is possible to isolate polynucleotide fragments that are, for example, at least 70% or at least 80% or at least from 90% to 95% identical with the sequence of the probe used. Further instructions for hybridization are commercially available in the form of so-called kits (e.g. DIG Easy Hyb from Roche Diagnostics GmbH, Mannheim, Germany, Catalog No. 1603558).

The person skilled in the art will find instructions on the amplification of DNA sequences with the aid of the polymerase chain reaction (PCR) inter alia in the handbook of Gait: Oligonukleotide synthesis: A Practical Approach (IRL Press, Oxford, UK, 1984) and in Newton and Graham: PCR (Spektrum Akademischer Verlag, Heidelberg, Germany, 1994).

It has been found that coryneform bacteria produce amino acids in an improved manner after enhancement of the rpoB gene.

In order to achieve overexpression, the number of copies of the corresponding genes can be increased, or the promoter and regulation region or the ribosome binding site, which is located upstream of the structural gene, can be mutated. Expression cassettes inserted upstream of the structural gene have a similar effect. By means of inducible promoters it is additionally possible to increase the expression in the course of the production of amino acids by fermentation. Expression is also improved by measures to prolong the life of the m-RNA. Furthermore, the enzyme activity is also enhanced by preventing degradation of the enzyme protein. The genes or gene constructs may either be present in plasmids with different numbers of copies or be integrated and amplified in the chromosome. Alternatively, overexpression of the genes in question may also be achieved by changing the composition of the medium and the manner in which culturing is carried out.

The person skilled in the art will find instructions thereon in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European patent specification 0 472 869, in U.S. Pat. No. 4,601,893, in Schwarzer and Pühler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in patent application WO 96/15246, in Malumbres et al. (Gene 134, 15-24 (1993)), in Japanese Offenlegungsschrift JP-A-10-229891, in Jensen and Hammer (Biotechnology and Bioengineering 58, 191-195 (1998)), in Makrides (Microbiological Reviews 60:512-538 (1996)) and in known textbooks of genetics and molecular biology.

For the purposes of enhancement, the rpoB gene of the invention was overexpressed, for example, with the aid of episomal plasmids. Suitable plasmids are those which are replicated in coryneform bacteria. Many known plasmid vectors, such as, for example, pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)), are based on the cryptic plasmids pHM1519, pBL1 or pGA1. Other plasmid vectors, such as, for example, those which are based on pCG4 (U.S. Pat. No. 4,489,160) or pNG2 (Serwold-Davis et al., FEMS Microbiology Letters 66, 119-124 (1990)) or pAG1 (U.S. Pat. No. 5,158,891), may likewise be used.

Also suitable are those plasmid vectors with the aid of which the process of gene amplification by integration into the chromosome can be applied, as has been described, for example, by Reinscheid et al. (Applied and Environmental Microbiology 60, 126-132 (1994)) for the duplication or amplification of the hom-thrB operon. In that method, the complete gene is cloned into a plasmid vector that is able to replicate in a host (typically E. coli), but not in C. glutamicum. Suitable vectors are, for example, pSUP301 (Simon et al., Bio/Technology 1, 784-791 (1983)), pK18mob or pK19mob (Schafer et al., Gene 145, 69-73 (1994)), PGEM-T (Promega corporation, Madison, Wis., USA), pCR2.1-TOPO (Shuman (1994). Journal of Biological Chemistry 269:32678-32684; U.S. Pat. No. 5,487,993), pCR®Blunt (Invitrogen, Groningen, Netherlands; Bernard et al., Journal of Molecular Biology, 234: 534-541 (1993)), pEM1 (Schrumpf et al., 1991, Journal of Bacteriology 173:4510-4516) or pBGS8 (Spratt et al., 1986, Gene 41: 337-342). The plasmid vector containing the gene to be amplified is then transferred to the desired strain of C. glutamicum by conjugation or transformation. The method of conjugation is described, for example, in Schafer et al. (Applied and Environmental Microbiology 60, 756-759 (1994)). Methods of transformation are described, for example, in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” occurrence, the resulting strain contains at least two copies of the gene in question.

It has also been found that the substitution of amino acids, especially in the sections between position 1 to 10, 190 to 200 and 420 to 450 in the amino acid sequence of the β-subunit of RNA polymerase B shown in SEQ ID No. 2, improves the lysine production of coryneform bacteria.

It has also been found that the substitution of amino acids at one or more positions selected from the group a) position 1 to 10, b) position 190 to 200 and c) position 420 to 450 in SEQ ID No. 2 may take place simultaneously.

In the region between position 1 to 10, preference is given to the substitution of L-proline at position 5 by L-leucine, L-isoleucine or L-valine.

In the region between position 190 to 200, preference is given to the substitution of L-serine at position 196 by L-phenylalanine or L-tyrosine.

In the region between 420 to 450, the following substitutions are preferred: substitution of L-leucine at position 424 by L-proline or L-arginine, substitution of L-serine at position 425 by L-threonine or L-alanine, substitution of L-glutamine at position 426 by L-leucine or L-lysine, substitution of L-aspartic acid at position 429 by L-isoleucine, L-valine or L-leucine, substitution of L-histidine at position 439 by any proteinogenic amino acid with the exception of L-histidine, is (sic) the substitution of L-serine at position 444 by L-leucine, L-tyrosine or L-tryptophan, and substitution of L-leucine at position 446 by L-proline or L-isoleucine.

Very special preference is given to one or more amino acid substitutions selected from the group: L-proline at position 5 by L-leucine, L-serine at position 196 by L-phenylalanine, L-aspartate at position 429 by L-valine, and L-histidine at position 439 by L-tyrosine.

SEQ ID No. 3 shows the base sequence of the allele rpoB-1547 contained in strain DM1547. The rpoB-1547 allele codes for a protein the amino acid sequence of which is shown in SEQ ID No. 4. The protein contains L-leucine at position 5, L-phenylalanine at position 196 and L-valine at position 429. The DNA sequence of the rpoB-1547 allele (SEQ ID No. 3) contains the following base substitutions as compared with the rpoB wild-type gene (SEQ ID No. 1): thymine at position 715 instead of cytosine, thymine at position 1288 instead of cytosine, and thymine at position 1987 instead of adenine.

SEQ ID No. 5 shows the base sequence of the allele rpoB-1546 contained in strain DM1546. The rpoB-1546 allele codes for a protein the amino acid sequence of which is shown in SEQ ID No. 6. The protein contains L-tyrosine at position 439. The DNA sequence of the rpoB-1546 allele (SEQ ID No. 5) contains the following base substitutions as compared with the rpoB wild-type gene (SEQ ID No. 1): thymine at position 2016 instead of cytosine.

There may be employed for the mutagenesis conventional methods of mutagenesis using mutagenic substances such as, for example, N-methyl-N′-nitro-N-nitrosoguanidine or ultraviolet light. There may also be used for the mutagenesis in vitro methods such as, for example, treatment with hydroxylamine (Miller, J. H.: A Short Course in Bacterial Genetics. A Laboratory Manual and Handbook for Escherichia coli and Related Bacteria, Cold Spring Harbor Laboratory Press, Cold Spring Harbor, 1992) or mutagenic oligonucleotides (T. A. Brown: Gentechnologie für Einsteiger, Spektrum Akademischer Verlag, Heidelberg, 1993) or the polymerase chain reaction (PCR), as is described in the handbook of Newton and Graham (PCR, Spektrum Akademischer Verlag, Heidelberg, 1994).

In addition, it may be advantageous for the production of L-amino acids to enhance, especially to overexpress, in addition to the rpoB gene, one or more enzymes of the biosynthesis pathway in question, of glycolysis, of the anaplerotic pathway, of the citric acid cycle, of the pentose phosphate cycle, of amino acid export, and, optionally, regulatory proteins.

Accordingly, for the production of L-lysine, in addition to enhancing the rpoB gene, one or more genes selected from the group

the gene dapA coding for dihydrodipicolinate synthase (EP-B 0 197 335),

the gene gap coding for glyceraldehyde 3-phosphate dehydrogenase (Eikmanns (1992), Journal of Bacteriology 174:6076-6086),

the gene tpi coding for triose phosphate isomerase (Eikmanns (1992), Journal of Bacteriology 174:6076-6086),

the gene pgk coding for 3-phosphoglycerate kinase (Eikmanns (1992), Journal of Bacteriology 174:6076-6086),

the gene zwf coding for glucose-6-phosphate dehydrogenase (JP-A-09224661),

the gene pyc coding for pyruvate carboxylase (DE-A-198 31 609),

the gene mqo coding for malate quinone oxidoreductase (Molenaar et al., European Journal of Biochemistry 254, 395-403 (1998)),

the gene lysc coding for a feed-back resistant aspartate kinase (Kalinowski et al., Molecular Microbiologie 5(5), 1197-1204 (1991)),

the gene lysE coding for lysine export (DE-A-195 48 222),

the gene zwa1 coding for the Zwa1 protein (DE: 19959328.0, DSM 13115), and

the rpsL gene coding for ribosomal protein S12 and shown in SEQ ID No. 7 and 8

may be enhanced, especially overexpressed.

The term “attenuation” in this connection describes the diminution or exclusion of the intracellular activity of one or more enzymes (proteins) in a microorganism that are coded for by the corresponding DNA, by, for example, using a weak promoter or using a gene or allele that codes for a corresponding enzyme having low activity, or by inactivating the corresponding gene or enzyme (protein), and optionally by combining those measures.

Furthermore, it may be advantageous for the production of L-amino acids, in addition to enhancing the rpoB gene, to attenuate, especially to diminish the expression of, one or more genes selected from the group

the gene pck coding for phosphoenol pyruvate carboxykinase (DE 199 50 409.1; DSM 13047),

the gene pgi coding for glucose-6-phosphate isomerase (U.S. Ser. NO. 09/396,478; DSM 12969),

the gene poxB coding for pyruvate oxidase (DE: 1995 1975.7; DSM 13114),

the gene zwa2 coding for the Zwa2 protein (DE: 19959327.2, DSM 13113).

It may also be advantageous for the production of amino acids, in addition to enhancing the rpoB gene, to exclude undesired secondary reactions (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms produced according to the invention also form part of the invention and can be cultivated, for the purposes of the production of amino acids, continuously or discontinuously in the batch, fed batch or repeated fed batch process. A summary of known cultivation methods is described in the textbook of Chmiel (Bioprozeβtechnik 1. Einführung in die Bioverfahrenstechnik (Gustav Fischer Verlag, Stuttgart, 1991)) or in the textbook of Storhas (Bioreaktoren und periphere Einrichtungen (Vieweg Verlag, Braunschweig/Wiesbaden, 1994)).

The culture medium to be used must meet the requirements of the strains in question in a suitable manner. Descriptions of culture media for various microorganisms are to be found in the handbook “Manual of Methods for General Bacteriology” of the American Society for Bacteriology (Washington D.C., USA, 1981).

There may be used as the carbon source sugars and carbohydrates, such as, for example, glucose, saccharose, lactose, fructose, maltose, molasses, starch and cellulose, oils and fats, such as, for example, soybean oil, sunflower oil, groundnut oil and coconut oil, fatty acids, such as, for example, palmitic acid, stearic acid and linoleic acid, alcohols, such as, for example, glycerol and ethanol, and organic acids, such as, for example, acetic acid. Those substances may be used individually or in the form of a mixture.

There may be used as the nitrogen source organic nitrogen-containing compounds, such as peptones, yeast extract, meat extract, malt extract, corn steep liquor, soybean flour and urea, or inorganic compounds, such as ammonium sulfate, ammonium chloride, ammonium phosphate, ammonium carbonate and ammonium nitrate. The nitrogen sources may be used individually or in the form of a mixture.

There may be used as the phosphorus source phosphoric acid, potassium dihydrogen phosphate or dipotassium hydrogen phosphate or the corresponding sodium-containing salts. The culture medium must also contain salts of metals, such as, for example, magnesium sulfate or iron sulfate, which are necessary for growth. Finally, essential growth substances, such as amino acids and vitamins, may be used in addition to the above-mentioned substances. Suitable precursors may also be added to the culture medium. The mentioned substances may be added to the culture in the form of a single batch, or they may be fed in in a suitable manner during the cultivation.

In order to control the pH value of the culture, basic compounds, such as sodium hydroxide, potassium hydroxide, ammonia or ammonia water, or acid compounds, such as phosphoric acid or sulfuric acid, are expediently used. In order to control the development of foam, anti-foams, such as, for example, fatty acid polyglycol esters, may be used. In order to maintain the stability of plasmids, suitable substances having a selective action, such as, for example, antibiotics, may be added to the medium. In order to maintain aerobic conditions, oxygen or gas mixtures containing oxygen, such as, for example, air, are introduced into the culture. The temperature of the culture is normally from 20° C. to 45° C. and preferably from 25° C. to 40° C. The culture is continued until the maximum amount of the desired product has formed. That aim is normally achieved within a period of from 10 hours to 160 hours.

Methods of determining L-amino acids are known from the prior art. The analysis may be carried out, for example, as described in Spackman et al. (Analytical Chemistry, 30, (1958), 1190) by ion-exchange chromatography with subsequent ninhydrin derivatization, or it may be carried out by reversed phase HPLC, as described in Lindroth et al. (Analytical Chemistry (1979) 51: 1167-1174).

Pure cultures of the following microorganisms were deposited on Jan. 16, 2001 at the Deutsche Sammlung für Mikroorganismen und Zellkulturen (DSMZ, Braunschweig, Germany) in accordance with the Budapest Treaty:

Corynebacterium glutamicum strain DM1546 as DSM 13993

Corynebacterium glutamicum strain DM1547 as DSM 13994.

The process of the invention is used for the production of amino acids by fermentation.

The present invention is explained in greater detail below by means of Examples.

The isolation of plasmid DNA from Escherichia coli and all techniques for restriction, Klenow and alkaline phosphatase treatment were carried out according to Sambrook et al. (Molecular Cloning. A Laboratory Manual (1989) Cold Spring Harbour Laboratory Press, Cold Spring Harbor, N.Y., USA). Methods for the transformation of Escherichia coli are also described in that handbook.

The composition of common nutrient media, such as LB or TY medium, will also be found in the handbook of Sambrook et al.

### EXAMPLE 1

Preparation of a Genomic Cosmid Gene Library from Corynebacterium glutamicum ATCC 13032

Chromosomal DNA from Corynebacterium glutamicum ATCC 13032 is isolated as described in Tauch et al. (1995, Plasmid 33:168-179) and partially cleaved with the restriction enzyme Sau3AI (Amersham Pharmacia, Freiburg, Germany, product description Sau3AI, Code no. 27-0913-02). The DNA fragments are dephosphorylated with shrimp alkaline phosphatase (Roche Diagnostics GmbH, Mannheim, Germany, product description SAP, Code no. 1758250). The DNA of cosmid vector SuperCosl (Wahl et al. (1987) Proceedings of the National Academy of Sciences USA 84:2160-2164), obtained from Stratagene (La Jolla, USA, product description SuperCosl Cosmid Vektor Kit, Code no. 251301), is cleaved with the restriction enzyme XbaI (Amersham Pharmacia, Freiburg, Germany, product description XbaI, Code no. 27-0948-02) and likewise dephosphorylated with shrimp alkaline phosphatase.

The cosmid DNA is then cleaved with the restriction enzyme BamHI (Amersham Pharmacia, Freiburg, Germany, product description BamHI, Code no. 27-0868-04). The cosmid DNA so treated is mixed with the treated ATCC13032 DNA, and the batch is treated with T4-DNA ligase (Amersham Pharmacia, Freiburg, Germany, product description T4-DNA ligase, Code no. 27-0870-04). The ligation mixture is then packed in phages with the aid of Gigapack II XL Packing Extract (Stratagene, La Jolla, USA, product description Gigapack II XL Packing Extract, Code no. 200217).

For infection of E. coli strain NM554 (Raleigh et al. 1988, Nucleic Acid Research 16:1563-1575), the cells are taken up in 10 mM MgSO₄ and mixed with an aliquot of the phage suspension. Infection and titration of the cosmid library are carried out as described in Sambrook et al. (1989, Molecular Cloning: A Laboratory Manual, Cold Spring Harbor), the cells being plated out on LB agar (Lennox, 1955, Virology, 1:190) with 100 mg/l ampicillin. After incubation overnight at 37° C., recombinant individual clones are selected.

### EXAMPLE 2

Isolation and Sequencing of the rpoB Gene

The cosmid DNA of an individual colony is isolated using the Qiaprep Spin Miniprep Kit (Product No. 27106, Qiagen, Hilden, Germany) according to the manufacturer's instructions, and partially cleaved with the restriction enzyme Sau3AI (Amersham Pharmacia, Freiburg, Germany, product description Sau3AI, Product No. 27-0913-02). The DNA fragments are dephosphorylated with shrimp alkaline phosphatase (Roche Diagnostics GmbH, Mannheim, Germany, product description SAP, Product No. 1758250). After separation by gel electrophoresis, cosmid fragments having a size in the range from 1500 to 2000 bp are isolated using the QiaExII Gel Extraction Kit (Product No. 20021, Qiagen, Hilden, Germany).

The DNA of sequencing vector pZero-1, obtained from Invitrogen (Groningen, Netherlands, product description Zero Background Cloning Kit, Product No. K2500-01), is cleaved with the restriction enzyme BamHI (Amersham Pharmacia, Freiburg, Germany, product description BamHI, Product No. 27-0868-04). Ligation of the cosmid fragments into the sequencing vector pZero-1 is carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). The ligation mixture is then electroporated into E. coli strain DH5αMCR (Grant, 1990, Proceedings of the National Academy of Sciences U.S.A., 87:4645-4649) (Tauch et al. 1994, FEMS Microbiol Letters, 123:343-347) and plated out on LB agar (Lennox, 1955, Virology, 1:190) with 50 mg/l Zeocin.

Plasmid preparation of the recombinant clones is carried out using the Biorobot 9600 (Product No. 900200, Qiagen, Hilden, Germany). Sequencing is effected by the dideoxy chain termination method of Sanger et al. (1977, Proceedings of the National Academy of Sciences U.S.A., 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) is used. Separation by gel electrophoresis and analysis of the sequencing reaction is carried out in a “Rotiphorese NF Acrylamid/Bisacrylamid” gel (29:1) (Product No. A124.1, Roth, Karlsruhe, Germany) using the “ABI Prism 377” sequencing device from PE Applied Biosystems (Weiterstadt, Germany).

The resulting crude sequence data are then processed using the Staden program package (1986, Nucleic Acids Research, 14:217-231) Version 97-0. The individual sequences of the pZero1 derivatives are assembled to a coherent contig. The computer-assisted coding region analysis is prepared using the program XNIP (Staden, 1986, Nucleic Acids Research, 14:217-231).

The resulting nucleotide sequence is shown in SEQ ID No. 1. Analysis of the nucleotide sequence gives an open reading frame of 3497 base pairs, which is designated the rpoB gene. The rpoB gene codes for a protein of 1165 amino acids.

Obviously, numerous modifications and variations on the present invention are possible in light of the above teachings. It is therefore to be understood that within the scope of the appended claims, the invention may be practiced otherwise than as specifically described herein.

1. An isolated polynucleotide which encodes a protein comprising the amino acid sequence of SEQ ID NO:2.

2. The isolated polynucleotide of claim 1, wherein said protein has the activity of the β-subunit of RNA polymerase B.

3. An isolated polynucleotide which comprises SEQ ID NO:1.

4. An isolated polynucleotide which is complimentary to the polynucleotide of claim 3.

5. An isolated polynucleotide which is at least 70% identical to the polynucleotide of claim 3.

6. An isolated polynucleotide which is at least 80% identical to the polynucleotide of claim 3.

7. An isolated polynucleotide which is at least 90% identical to the polynucleotide of claim 3.

8. An isolated polynucleotide which hybridizes under stringent conditions to the polynucleotide of claim 3; wherein said stringent conditions comprise washing in 5×SSC at a temperature from 50 to 68° C.

9. The isolated polynucleotide of claim 3, which encodes a protein having the activity of the β-subunit of RNA polymerase B.

10. An isolated polynucleotide which comprises at least 15 consecutive nucleotides of the polynucleotide of claim 3.

11. An isolated polypeptide which comprises the amino acid sequence of SEQ ID NO:2.

12. An isolated polypeptide which comprises the amino acid sequence of SEQ ID NO:4.

13. An isolated polypeptide which comprises the amino acid sequence of SEQ ID NO:6.

14. An isolated polynucleotide which encodes a protein comprising the amino acid sequence of SEQ ID NO:4.

15. An isolated polynucleotide which comprises SEQ ID NO:3.

16. An isolated polynucleotide which encodes a protein comprising the amino acid sequence of SEQ ID NO:6.

17. An isolated polynucleotide which comprises SEQ ID NO:5.

18. A vector comprising the isolated polynucleotide of claim 1.

19. A vector comprising the isolated polynucleotide of claim 3.

20. A vector comprising the isolated polynucleotide of claim 14.

21. A vector comprising the isolated polynucleotide of claim 15.

22. A vector comprising the isolated polynucleotide of claim 16.

23. A vector comprising the isolated polynucleotide of claim 17.

24. A host cell comprising the isolated polynucleotide of claim 1.

25. A host cell comprising the isolated polynucleotide of claim 3.

26. A host cell comprising the isolated polyncleotide of claim 14.

27. A host cell comprising the isolated polyncleotide of claim 15.

28. A host cell comprising the isolated polyncleotide of claim 16.

29. A host cell comprising the isolated polyncleotide of claim 17.

30. The host cell of claim 24, which is a Coryneform bacterium.

31. The host cell of claim 25, which is a Coryneform bacterium.

32. The host cell of claim 26, which is a Coryneform bacterium.

33. The host cell of claim 27, which is a Coryneform bacterium.

34. The host cell of claim 28, which is a Coryneform bacterium.

35. The host cell of claim 29, which is a Coryneform bacterium.

36. The host cell of claim 24, wherein said host cell is selected from the group consisting of Coryneform glutamicum, Corynebacterium acetoglutamicum, Corynebacterium acetoacidophilum, Corynebacterium thermoaminogenes, Corynebacterium melassecola, Brevibacterium flavum, Brevibacterium lactofermentum, and Brevibacterium divaricatum.

37. The host cell of claim 24, wherein said host cell is selected from the group consisting of Corynebacterium glutamicum FERM 1709, Brevibacterium flavum FERM-P 1708, Brevibacterium lactofermentum FERM-P1712, Corynebacterium glutamicum FERM-P6463, Corynebacterium glutamicum FERM-P6464, Corynebacterium glutamicum DM58-1, Corynebacterium glutamicum DG 52-5, Corynebacterium glutamicum DSM 5714 and Corynebacterium glutamicum DSM-12866.

38. The host cell of claim 25, wherein said host cell is selected from the group consisting of Coryneform glutamicum, Corynebacterium acetoglutamicum, Corynebacterium acetoacidophilum, Corynebacterium thermoaminogenes, Corynebacterium melassecola, Brevibacterium flavum, Brevibacterium lactofermentum, and Brevibacterium divaricatum.

39. The host cell of claim 25, wherein said host cell is selected from the group consisting of Corynebacterium glutamicum FERM 1709, Brevibacterium flavum FERM-P 1708, Brevibacterium.lactofermentum FERM-P1712, Corynebacterium glutamicum FERM-P6463, Corynebacterium glutamicum FERM-P6464, Corynebacterium glutamicum DM58-1, Corynebacterium glutamicum DG 52-5, Corynebacterium glutamicum DSM 5714 and Corynebacterium glutamicum DSM-12866.

40. A Coryneform bacterium which comprises an enhanced rpoB gene.

41. The Coryneform bacterium of claim 40, wherein said rpoB gene comprises the polynucleotide sequence of SEQ ID NO:1.

42. The Coryneform bacterium of claim 40, wherein said enhanced rpoB gene comprises the polynucleotide sequence of SEQ ID NO:3.

43. The Coryneform bacterium of claim 40, wherein said enhanced rpoB gene comprises the polynucleotide sequence of SEQ ID NO:5.

44. Coryneform glutamicum DSM 13993.

45. Coryneform glutamicum DSM 13994.

46. A process for producing L-amino acids comprising culturing the host cell of claim 24 in a medium suitable for the expression of the polynucleotide; and collecting the L-amino acid.

47. The process of claim 46, wherein said L-amino acid is L-lysine or L-glutamate.

48. The process of claim 46, wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwa1 and rpsl.

49. The process of claim 46, wherein the host cell further comprises at least one gene whose expression is attenuated, wherein said gene is selected from the group consisting of pck gene, pgi gene, poxB, and zwa2.

50. A process for producing L-amino acids comprising culturing the host cell of claim 25 in a medium suitable for the expression of the polynucleotide; and collecting the L-amino acid.

51. The process of claim 50, wherein said L-amino acid is L-lysine or L-glutamate.

52. The process of claim 50, wherein wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwa1 and rpsL.

53. The process of claim 50, wherein the host cell further comprises at least one gene whose expression is attenuated, wherein said gene is selected from the group consisting of pck gene, pgi gene, poxB, and zwa2.

54. A process for producing L-amino acids comprising culturing the host cell of claim 26 in a medium suitable for the expression of the polynucleotide; and collecting the L-amino acid.

55. The process of claim 54, wherein said L-amino acid is L-lysine or L-glutamate.

56. The process of claim 54, wherein wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwal and rpsL.

57. The process of claim 54, wherein the host cell further comprises at least one gene whose expression is attenuated, wherein said gene is selected from the group consisting of pck gene, pgi gene, poxB, and zwa2.

58. A process for producing L-amino acids comprising culturing the host cell of claim 26 in a medium suitable for the expression of the polynucleotide; and collecting the L-amino acid.

59. The process of claim 58, wherein said L-amino acid is L-lysine or L-glutamate.

60. The process of claim 58, wherein wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwa1 and rpsL.

61. The process of claim 58, wherein the host cell further comprises at least one gene whose expression is attenuated, wherein said gene is selected from the group consisting of pck gene, pgi gene, poxB, and zwa2.

62. A process for producing L-amino acids comprising culturing the host cell of claim 27 in a medium suitable for the expression of the polynucleotide; and collecting the L-amino acid.

63. The process of claim 62, wherein said L-amino acid is L-lysine or L-glutamate.

64. The process of claim 62, wherein wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwa1 and rpsL.

65. The process of claim 62, wherein the host cell further comprises at least one gene whose expression is attenuated, wherein said gene is selected from the group consisting of pck gene, pgi gene, poxB, and zwa2.

66. A process for producing L-amino acids comprising culturing the host cell of claim 28 in a medium suitable for the expression of the polynucleotide; and collecting the L-amino acid.

67. The process of claim 66, wherein said L-amino acid is L-lysine or L-glutamate.

68. The process of claim 66, wherein wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwa1 and rpsL.

69. The process of claim 66, wherein the host cell further comprises at least one gene whose expression is attenuated, wherein said gene is selected from the group consisting of pck gene, pgi gene, poxB, and zwa2.

70. A process for producing L-amino acids comprising culturing the host cell of claim 29 in a medium suitable for the expression of the polynucleotide; and collecting the L-amino acid.

71. The process of claim 70, wherein said L-amino acid is L-lysine or L-glutamate.

72. The process of claim 70, wherein wherein said L-amino acid is L-lysine and the host cell further comprises at least one gene whose expression is enhanced, wherein said gene is selected from the group consisting of dapA, gap, tpi, pgk, zwf, pyc, mqo, lys C, lys E, zwa1 and rpsL.

73. The process of claim 70, wherein the host cell further comprises at least one gene whose expression is attenuated, wherein said gene is selected from the group consisting of pck gene, pgi gene, poxB, and zwa2.

74. A process for screening for polynucleotides which encode a protein having the activity of the β-subunit of RNA polymerase B comprising hybridizing the isolated polynucleotide of claim 1 to the polynucleotide to be screened; expressing the polynucleotide to produce a protein; and detecting the presence or absence of the activity of the β-subunit of RNA polymerase B in said protein.

75. A process for screening for polynucleotides which encode a protein having the activity of the β-subunit of RNA polymerase B comprising hybridizing the isolated polynucleotide of claim 3 to the polynucleotide to be screened; expressing the polynucleotide to produce a protein; and detecting the presence or absence of the activity of the β-subunit of RNA polymerase B in said protein.

76. A process for screening for polynucleotides which encode a protein having the activity of the β-subunit of RNA polymerase B comprising hybridizing the isolated polynucleotide of claim 15 to the polynucleotide to be screened; expressing the polynucleotide to produce a protein; and detecting the presence or absence the activity of the β-subunit of RNA polymerase B in said protein.

77. A process for screening for polynucleotides which encode a protein having the activity of the β-subunit of RNA polymerase B comprising hybridizing the isolated polynucleotide of claim 17 to the polynucleotide to be screened; expressing the polynucleotide to produce a protein; and detecting the presence or absence the activity of the β-subunit of RNA polymerase B in said protein.

78. A method for detecting a nucleic acid with at least 70% homology to nucleotide of claim 1, comprising contacting a nucleic acid sample with a probe or primer comprising at least 15 consecutive nucleotides of the nucleotide sequence of claim 1, or at least 15 consecutive nucleotides of the complement thereof.

79. A method for producing a nucleic acid with at least 70% homology to nucleotide of claim 1, comprising contacting a nucleic acid sample with a primer comprising at least 15 consecutive nucleotides of the nucleotide sequence of claim 1, or at least 15 consecutive nucleotides of the complement thereof.

80. A method for detecting a nucleic acid with at least 70% homology to nucleotide of claim 3, comprising contacting a nucleic acid sample with a probe or primer comprising at least 15 consecutive nucleotides of the nucleotide sequence of claim 3, or at least 15 consecutive nucleotides of the complement thereof.

81. A method for producing a nucleic acid with at least 70% homology to nucleotide of claim 3, comprising contacting a nucleic acid sample with a primer comprising at least 15 consecutive nucleotides of the nucleotide sequence of claim 3, or at least 15 consecutive nucleotides of the complement thereof.

82. A method for making a β-subunit of RNA polymerase B, comprising: culturing the host cell of claim 23 for a time and under conditions suitable for expression of the β-subunit of RNA polymerase B, and collecting the β-subunit of RNA polymerase B.

83. A method for making a β-subunit of RNA polymerase B, comprising: culturing the host cell of claim 24 for a time and under conditions suitable for expression of the β-subunit of RNA polymerase B, and collecting the β-subunit of RNA polymerase B.

84. A method for making a β-subunit of RNA polymerase B, comprising: culturing the host cell of claim 25 for a time and under conditions suitable for expression of the β-subunit of RNA polymerase B, and collecting the β-subunit of RNA polymerase B.

85. A method for making a β-subunit of RNA polymerase B, comprising: culturing the host cell of claim 26 for a time and under conditions suitable for expression of the β-subunit of RNA polymerase B, and collecting the β-subunit of RNA polymerase B.

86. A method for making a β-subunit of RNA polymerase B, comprising: culturing the host cell of claim 27 for a time and under conditions suitable for expression of the β-subunit of RNA polymerase B, and collecting the β-subunit of RNA polymerase B.

87. A method for making a β-subunit of RNA polymerase B, comprising: culturing the host cell of claim 28 for a time and under conditions suitable for expression of the β-subunit of RNA polymerase B, and collecting the β-subunit of RNA polymerase B.
